(19) United States
(12) Patent Application Publication
(10) Pub. No.: US 2015/0374940 A1
(43) Pub. Date: Dec. 31, 2015

(54) System and Method for Acclimating A New User To A Breathing Therapy Machine

(71) Applicant: DeVilbiss Healthcare LLC, Somerset, PA (US)

(72) Inventors: James P. Froehlich, Berlin, PA (US); Brian Palmer, New Albany, OH (US)

(21) Appl. No.: 14/316,373

(22) Filed: Jun. 26, 2014

(51) Int. Cl. A61M 16/00; A61M 16/20

(52) U.S. Cl. CPC A61M 16/0066; A61M 16/0003; A61M 16/208; A61M 2016/0027; A61M 2016/003; A61M 2016/0015; A61M 2205/3334; A61M 2205/502

(57) ABSTRACT

A method for acclimating a new user to a CPAP or BiPAP breathing therapy device in which the pressure delivered to the patient increases from an initial, starting pressure to the desired therapy pressure in a linear manner over the course of several sleep sessions, with the start pressure for each successive sleep session starting at the ending pressure for the previous sleep session.

### FIELD OF THE INVENTION

This invention is related to the field of breathing gas delivery machines, such as continuous positive airway pressure (CPAP) or bi-level positive airway pressure (Bi-PAP) machines of the type typically used to treat patients suffering from breathing disorders, such as hypopnea or apnea, and, in particular, is related to a method of acclimating new users of the machines during the initial phase of use.

### BACKGROUND OF THE INVENTION

Continuous Positive Airways Pressure (CPAP) machines are well known in the art for use in the treatment of a number of respiratory conditions, such as sleep apnea and hypopnea, by supplying a continuous positive pressure to a patient's airway while the patient sleeps. A typical CPAP apparatus is programmed with a CPAP therapy pressure, and is able to maintain the set pressure (measured either at the mask or at a base unit) during the inhalation and exhalation phases of the breathing cycle.

In a variation of the basic CPAP machine, two air pressure levels are able to be programmed into the machine, an inspiratory positive airway pressure (IPAP), delivered during the inhalation phase of the breathing cycle, and a expiratory positive airway pressure (EPAP), delivered during the exhalation phase of the breathing cycle. This type of machine is often referred to as a Bi-Level machine or BiPAP.

New users of both CPAP and BiPAP machines often experience discomfort prior to becoming accustomed to using the machine. The delivery of pressurized air to the airway of the patient produces an uncomfortable sensation due to the increased work required to breathe against the applied pressure. It is common for new patients to complain about the discomfort that is associated with the delivered pressure at the prescribed pressure level. In some cases the prescribed pressure setting is higher than what the patient can tolerate. This discomfort often causes the new user to cease use of the machine before any benefit can be realized. It has been found that as many as 20% to 40% of patients for which breathing therapy is prescribed fail to adhere to the prescribed therapy due to this discomfort.

It would therefore be advantageous to provide a feature in the machine to allow new users to become gently acclimated to the new sensations produced by the use of the machine over a period of days. This would likely increase patient compliance with the prescribed therapy.

Several attempts have been made to provide this feature in the prior art. U.S. Published Patent Application 2009/0293875 (Dec. 3, 2009) by Kwok, et al. describes one such method. In the primary embodiment, the pressure is increased by a pre-determined increment over several sleep sessions until the desired therapeutic pressure is reached. In an alternate embodiment, the pressure is started at a lower pressure than the increment, and ramped up to the increment for that sleep session, wherein it levels off for the remainder of that sleep session.

Published U.S. Patent Application 2010/0095959 (Apr. 22, 2010) by Farrell describes another method of acclimating a new patient in which the initial pressure for each sleep session is started at the same pressure and ramped up over the first few minutes of the sleep session to a higher pressure, at which point the pressure profile levels off to a predetermined pressure for that sleep session. The pre-determined pressure for each successive sleep session is raised until the desired therapeutic pressure is reached.

Each of these methods presents a sub-optimal solution to the problem. In the methods of both Kwok and Farrell, the delivered pressure profiles have flat areas which delay the user's acceptance of the discomfort associated with the use of the machine. Therefore, it would be desirable to have a solution that eliminated these deficiencies.

### SUMMARY OF THE INVENTION

The present invention presents what the inventors believe to be an optimal method of acclimating a new patient to the use of a CPAP or BiPAP breathing therapy device. The acclimation feature is intended to provide a delivered pressure which is lower than the prescribed pressure for a pre-determined period of time, the acclimation period, which may include several sleep sessions. During the acclimation period, the pressure will automatically be slowly increased in a ramped fashion. The ever-increasing pressure will likely go unnoticed by the patient and, over the duration of the acclimation period, the patient will develop a tolerance for higher and higher pressures, until the prescribed pressure is reached.

In the preferred embodiment, the pressure is constantly ramped up at a pre-determined rate, regardless of how long a sleep session lasts. This is repeated over several successive sleep sessions, with the pressure at each successive sleep session starting at the pressure where the previous sleep session left off. This continues for a period of acclimation having a pre-determined number of hours, regardless of how many sleep sessions are necessary. The number of sleep sessions required to complete the acclimation period will be determined by the length of the sleep sessions. The ramp rate during each sleep session is calculated by determining the total rise in pressure from the lowest pressure (i.e., the pressure where the first sleep session starts) and the desired therapeutic pressure, and dividing it by the total number of pre-determined hours in the acclimation period.

This method eliminates the “flat” areas in the delivered pressure profile that is a deficiency of other prior art methods. In the method of the present invention, the pressure is constantly rising, albeit at a slow rate, over a long period of time. It has been found that this method of acclimation increases patient acceptance of the device as the changes in delivered pressure are too small for the patient to notice, but accumulate over time to get the patient to the desired therapeutic pressure.

### BRIEF DESCRIPTION OF THE DRAWINGS

FIG. 1 is a schematic depiction of a typical prior art CPAP or BiPAP machine of the type that would be employed with the present invention

FIG. 2 is a flow diagram of the method of the present invention.

FIG. 3 illustrates the pressure profile of the device in accordance with the present invention.

### DETAILED DESCRIPTION OF THE INVENTION

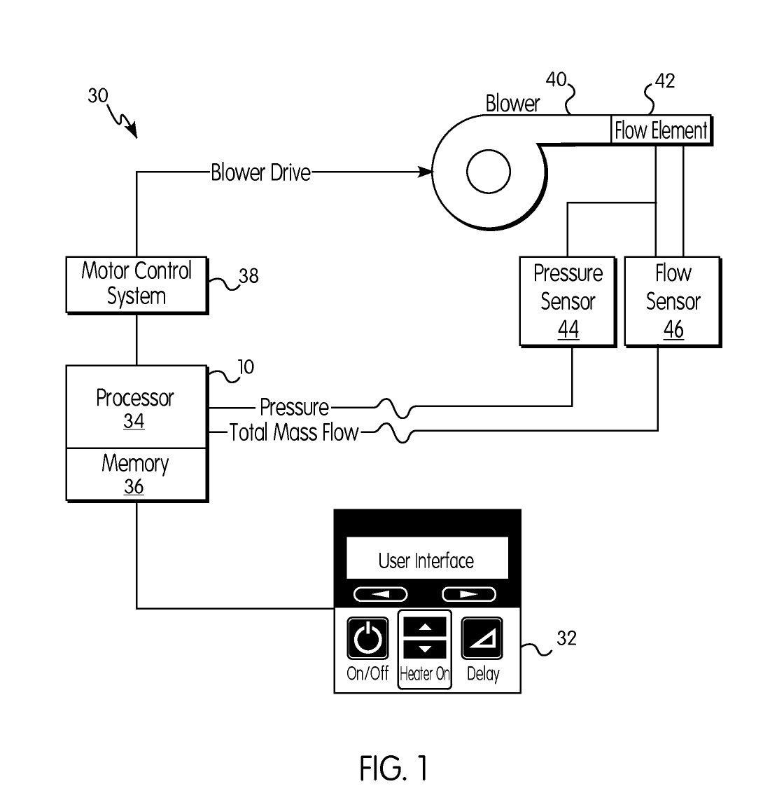

FIG. 1 is a block diagram of a typical breathing therapy device on which the present invention could be implemented. The device 30 includes a main unit housing microprocessor 34 and related non-volatile memory 36, as well as blower 40 and motor control circuitry 38. The device 30 is typically equipped with a user interface panel 32 which may be used to control the device as well as to program various operating parameters into the device.

Microprocessor 34 runs software stored in non-volatile memory 36, which implements the algorithms controlling blower 40 to regulate the pressure being delivered to the user of the device. The acclimation method of the present invention could be implemented as a control algorithm stored as software in non-volatile memory 36 and executed by microprocessor 34.

Microprocessor 34 and non-volatile memory 36 control the functioning of the unit. In one possible alternative embodiment, the unit may be provided with a separate microprocessor or Application Specific Integrated Circuit (ASIC) dedicated to the collection, storage and manipulation of the system software, parameters and selected operating data (not shown). Such dedicated components may use either the original memory hardware 36 or a separate dedicated memory for storing the data. Memory for the storage of operating algorithms and collected data can include any type of memory well known in the art, including non-volatile RAM, flash media and hard drives, any of which may be internal or external to the device.

Microprocessor 34 is electrically connected to motor control circuitry 38 that, in turn, is electrically connected to blower 40. Blower 40 provides pressurized breathing gas to a patient interface (not shown) via flow element 42. Motor control circuitry 38 is operative to control the speed of blower 40, and, thus, the pressure and flow rate of the air forced into flow element 42. Alternatively, pressure and flow rate may be controlled by a check valve disposed within flow element 42.

Pressure sensor 44 and flow sensor 46 are provided to monitor the pressure and flow rate, respectively, of the air passing through flow element 42 and to provide that information to microprocessor 34. Feedback voltages representing the blower motor current, the air pressure and the air flow rate are supplied by motor control circuitry 38, pressure sensor 44 and flow sensor 46, respectively, to an analog to digital converter (not shown) that converts the data to a digital format for use in control of device 30.

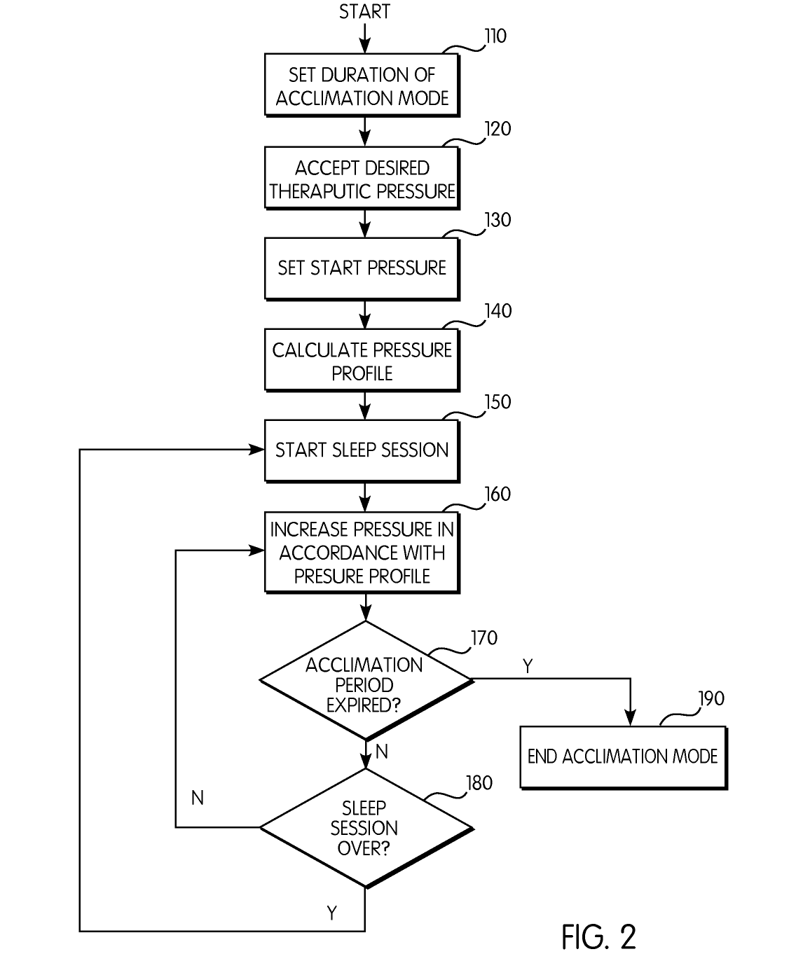

The steps of the method implementing the acclimation mode will now be discussed with reference to the flow chart in FIG. 2. In step 110 of the method, the duration of the acclimation mode may be set. In the preferred embodiment of the invention, this parameter has a default value of fifty hours, however, it may be that the prescribing physician or therapist has determined that another duration for the acclimation mode is desirable for a particular patient, in which case the duration may be input as one of the prescribed parameters before the device is delivered to the user.

In step 120, the prescribed therapeutic pressure 220 is set. Again, this value is user dependent and is a prescribed variable which is set in the machine prior to its delivery to the patient.

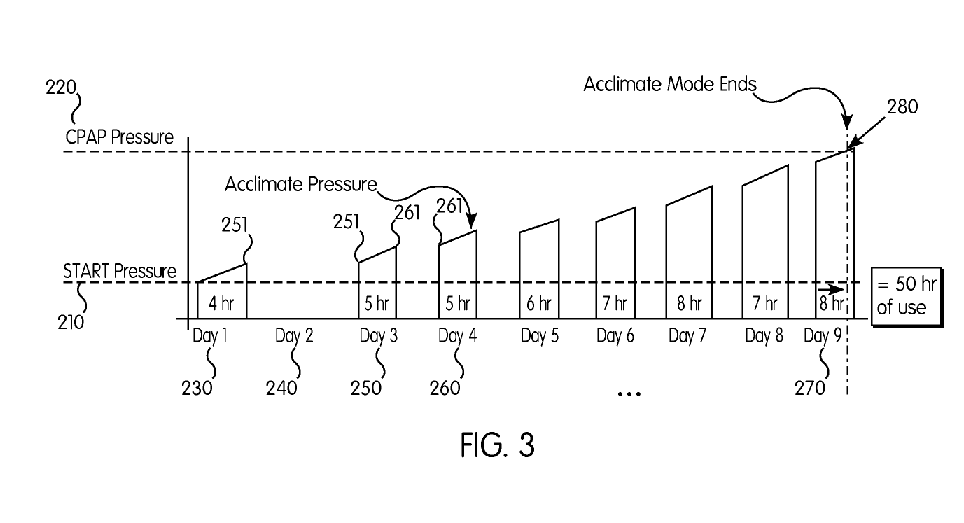

In step 130, the start pressure 210 is set. The start pressure is the pressure at which the acclimation mode begins and is shown in FIG. 3 as reference number 210. This pressure may also be set to a default value if not prescribed by the physician or therapist. In the preferred embodiment of the invention, the start pressure defaults to a value of 4 cm-H₂O, but this parameter may otherwise be entered by the prescribing physician or therapist using user interface 32.

In step 140, the pressure profile is calculated. In the preferred embodiment of the invention, the pressure profile is linear, however, in other embodiments of the invention, the pressure profile may be non-linear and calculated as a mathematical function of start pressure 210, the desire therapeutic pressure 220 and the duration of the acclimation period. In preferred embodiments of the invention, the mathematical function may be built into the machine and selectable by the prescribing physician or therapist, or may be input into the machine prior to its delivery to the patient.

The pressure profile determines the rate of increase in the pressure over time starting at the start pressure 210 and increasing to the desired therapeutic pressure 220 over the period of the duration of the acclimation mode. If the prescribing physician or therapist decides to use the default values for acclimation mode, then the only prescribed variable which needs to be set (in addition to the desired therapeutic pressure 220) is the one time setting enabling the acclimation mode to run one or more times prior to the machine being delivered to the patient.

The acclimation mode starts in step 150, at the start of a sleep session. Typically, a sleep session will be initiated by the user utilizing the user controls on user interface panel 32 or by simply turning the machine on. In the preferred embodiment, during each sleep session, the pressure delivered to the patient is increased in step 160 in accordance with the pressure profile which was calculated in step 140. The pressure is increased starting at the start pressure 210 and ending at the desire therapeutic pressure 220 over a period specified by the duration of the acclimate mode, which may include several sleep sessions.

An exemplary acclimation period is shown in FIG. 3. As can be seen in FIG. 3, the first sleep session on day 1 (230) has a duration of 4 hours, during which the delivered pressure increases from the start pressure 210 to a value dependent upon the pressure profile calculated in step 140 and the duration of the sleep session 230. It should be noted that FIG. 3 shows the preferred embodiment of the invention in which the pressure profile is a straight line.

In step 170, it is determined if the acclimation period has expired and, if not, in step 180, it is determined if the sleep session is over. If the sleep session is still continuing, which can be determined either by detection of various parameters, using the sensors of the machine or by the user turning the machine off, control returns to step 160 where the pressure continues to be increased in accordance with the pressure profile (in this example in a linear fashion). If, in step 180, it has been determined that the sleep session is over, control returns to step 150, where the machine awaits for the start of the next sleep session.

Referring again to FIG. 3, it will be noted that on day 2 (240) the patient has skipped the use of the machine and on day 3 (250) the user has slept for a total of five hours. It should be noted that at the beginning pressure 251 of the sleep session on day 3 (250) is the same as the pressure at the end of the day 1 sleep session 230. Thus, each successive sleep session starts at a point on the pressure profile where the previous sleep session left off. Likewise, at the beginning of the day 4 sleep session 260 the beginning pressure 261 is the same as the ending pressure at the completion of the sleep session on day 3 (250). This continues on successive days until the duration of the acclimation mode is expired.

Referring now to step 170, if, during the sleep session, the acclimation period has expired (i.e., the specified duration has been reached), control shifts to step 190 where the acclimation mode ends and regular operation of the machine begins. At this point the pressure setting should be at the desired therapeutic pressure 220. This is also shown on day 9 (270) in FIG. 3 where the acclimation mode has ended at the point referred to with reference number 280 at which time the pressure, which has been linearly increasing since day 1 (230) of the acclimation mode has reached the desired therapeutic pressure 220 as prescribed by the physician or therapist.

It should be noted that while the invention has been explained with reference to a CPAP machine having a single therapeutic pressure. The application is equally applicable to a bi-level PAP machine having an inhalation pressure setting and an exhalation pressure setting. In the case of a bi-level PAP machine, the inhalation pressure is treated as the desired therapeutic pressure 220 and follows the pressure profile. The changes in the exhalation pressure follow the changes in the inhalation pressure, keeping a constant difference between the inhalation pressure and the exhalation pressure. For example, if a physician or therapist has prescribed a difference between the inhalation pressure and the exhalation pressure of 6 cm-H₂O, then that gap will remain constant throughout the acclimation period, unless the initial start pressure 210 is below the difference between the inhalation pressure and the exhalation pressure, in which case the exhalation pressure will be set to 0 until the inhalation pressure has increased sufficiently to allow the prescribed gap. Thereafter, the gap between the inhalation pressure and the exhalation pressure should be held constant by the machine, as the inhalation pressure rises to the desired therapeutic pressure 220.

In alternate embodiments of the invention, as previously discussed, the duration of the acclimation mode could be a variable time selectable by the physician or patient. In other alternate embodiments of the invention, various options could be implemented, including allowing the patient to reset the acclimation mode to run multiple times. In yet another embodiment of the invention, the acclimation mode may use a different mathematical function to produce a pressure profile between starting pressure 210 and ending pressure 220. For example, the pressure profile may have a slight curvature.

Lastly, the breathing therapy machine may be equipped with an auto adjust function which will work in concert with the acclimation mode feature. The auto adjust function allows the breathing therapy device to respond to apnea, hyponia and snoring events by raising the pressure to suppress the occurrence of the events. The auto adjust function decreases the current pressure setting by a small amount periodically in the absence of events. When an event is detected, the current pressure setting is raised. In acclimation mode, the pressure will never be raised above the pressure dictated by the pressure profile.

I claim:

1. A method for acclimating a user to a breathing therapy machine capable of delivering air to the user at varying pressures, comprising the steps of:
a. calculating a pressure profile for an acclimation period based on a desired therapeutic pressure, a start pressure and a duration of said acclimation period; and
b. delivering pressurized air to said user in accordance with said pressure profile over a plurality of successive sleep sessions for the duration of said acclimation period;
c. wherein the start pressure for each successive sleep session is the same as the ending pressure for the immediately preceding sleep session.

2. The method of claim 1 wherein said step of calculating a pressure profile comprises applying a mathematical function over said duration of said acclimation period.

3. The method claim 2 wherein said mathematical function results in a linear pressure profile.

4. The method of claim 1 wherein said duration of said acclimation period may be set.

5. The method of claim 1 wherein said duration of said acclimation period is a default value stored within said breathing therapy machine.

6. The method of claim 1 wherein said start pressure may be set.

7. The method of claim 1 wherein said start pressure is a default value stored within said breathing therapy machine.

8. The method of claim 1 wherein said breathing therapy machine is a bi-level machine having an inhalation pressure setting and an exhalation pressure setting and further wherein said inhalation pressure setting is controlled by said pressure profile.

9. The method of claim 8 wherein said changes in said exhalation pressure tracks changes in said inhalation pressure.

10. The method of claim 1 wherein said user may re-start said acclimation period.

11. A breathing therapy device capable of delivering air to a user at varying pressures, said device implementing an algorithm comprising the steps of:
a. a blower;
b. a processor;
c. a motor control component, under control of said processor, for controlling the speed of said blower;
d. a user interface; and
e. software, executed by said processor to provide the following functions:
i. calculating a pressure profile for an acclimation period based on a desired therapeutic pressure, a start pressure and a duration of said acclimation period; and
ii. delivering pressurized air to said user in accordance with said pressure profile over a plurality of successive sleep sessions for the duration of said acclimation period;
iii. wherein the start pressure for each successive sleep session is the same as the ending pressure for the immediately preceding sleep session.

12. The device of claim 11 wherein said pressure profile is created using a mathematical function.

13. The device of claim 11 wherein said values for said start pressure and said duration may be entered via said user interface.

14. The device of claim 11 wherein said values for said start pressure, said duration and said desired therapeutic pressure are set prior to delivery of said device to said user.

15. The device of claim 11 wherein said device is a bi-level breathing therapy machine having an inhalation pressure setting and an exhalation pressure setting and further wherein said software provides the further function of setting said inhalation pressure setting in conformance with said pressure profile, wherein said changes in said exhalation pressure track changes in said inhalation pressure.
